(19) United States
(12) Patent Application Publication
(10) Pub. No.: US 2009/0129983 A1
(43) Pub. Date: May 21, 2009

(54) Device For the Growth of Macromolecular Crystals and Drug Screening

(76) Inventors: Oscar J. Carlton IV, Huntsville, AL (US); Scott G. Hardin, Alabaster, AL (US); Kriss Houghland, Huntsville, AL (US); Thomas P. Lewis, Birmingham, AL (US); Joseph D. Ng, Huntsville, AL (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/912,445

(22) PCT Filed: Apr. 28, 2006
(86) PCT No.: PCT/US06/16453

§ 371 Date: Feb. 28, 2008

Related U.S. Application Data
(60) Provisional application No. 60/676,147, filed on Apr. 29, 2005.

(51) Int. Cl. G01N 21/00; B01L 3/00; C12M 1/00

(52) U.S. Cl. 422/82.05; 422/99; 435/283.1

(57) ABSTRACT

The invention is a device for counter-diffusion applications comprising a removable cartridge having a plurality of capillary tubes that may be disposed between first and second members. The first member may be moveable into at least a first and second position. The second member may be moveable into a sealing position wherein the distal ends of the capillary tubes contact a sealant material. In the first position, the proximal ends of the capillary tubes may contact a macromolecular solution, which may cause the macromolecular solution to diffuse into the interior space of the capillary tube. In the second position, the proximal ends of the capillary tubes may be inserted into a corresponding reservoir well having a precipitating solution. The macromolecular solution and the precipitating solution may then counter diffuse against each other in each capillary tube. The removable cartridge may then be removed and replaced with a new removable cartridge.

### FEDERALLY SPONSORED RESEARCH OR DEVELOPMENT

The United States Government may have rights in the inventions set forth herein as provided by the terms of Cooperative Agreement NCC8-246 awarded by the National Aeronautics and Space Administration.

### BACKGROUND OF THE INVENTION

The recent deciphering of entire genomic sequences of different organisms, including humans, has resulted in a demand to decipher three-dimensional structures of protein gene products. Determining the structures of proteins may allow researchers to compile structural information that may help facilitate predictions of function for almost any protein from knowing its coding sequences. Gaining a better understanding of protein structure and function may enable drug researchers to develop new drug treatments that target specific human, animal, and plant diseases. The human body alone has an estimated 52,000 different proteins. Determining the structures to atomic resolution for all these proteins is a daunting challenge, at best. X-ray crystallography currently offers one method to achieve this goal and is the only method to date for determining macromolecules greater than 35,000 Daltons.

Advanced recombinant DNA methods, systematic approaches for protein crystallization, and highly developed X-ray diffraction instruments and procedures have been developed to help improve the process for determining protein structure.

Although such advances have generally improved the rate and efficiency at which the crystals can be analyzed, the ability to obtain protein crystals that are suitable for X-ray diffraction remains a limiting-step.

A common approach in determining the structure of proteins is to consolidate crystallographic procedures to obtain as many protein crystals as feasible, determine their three-dimensional structure as quickly as possible, and eventually determine the function of each protein. In some cases, only those proteins that can be more easily obtained and purified are studied, while proteins that may be more difficult to crystallize may be reserved for later study. As a result, the actual number of protein structures that have been resolved may be less than 10% of the total number of proteins that have been cloned, expressed, and purified.

The general strategy for protein crystallization is to reduce the solubility of the macromolecules to produce a supersaturation state with respect to the protein. The supersaturation state of the protein may result in the formation of crystals. The parameters under which protein crystals are formed may be specific to the particular protein being studied. As a result, methods for growing protein crystals may require trial and error to determine which crystallization parameters may result in the growth of protein crystals. Such parameters may include pH, ionic strength, salt concentration in the precipitant, temperature, gravity, and viscosity, to name but a few. To determine which parameters may result in crystal growth, a single protein solution may be screened against multiple different screening parameters. Under conventional screening methods, each crystallization parameter is typically screened against only one parameter at a time. Once a useful parameter is initially identified, the parameter may be further optimized to produce crystals that may be useful for X-ray diffraction.

Batch crystallization and vapor diffusion crystallization are two common conventional techniques that may be used to obtain protein crystals. In batch crystallization, an undersaturated protein solution is mixed with a precipitant solution to change the solubility of the protein within the solution. The change in solubility may cause the protein solution to become supersaturated. Vapor diffusion crystallization involves mixing a protein solution and a precipitant solution together in a sitting or hanging drop supported by some surface and set to equilibrate against a reservoir of precipitant solution within a closed chamber. As water or other volatile components within the protein droplet are equilibrated with the reservoir, the protein and precipitant droplet become concentrated driving the system to supersaturation. In both techniques, a protein crystal may result if the chemical and physical parameters of the precipitant solution are well chosen. Such parameters may include pH, ionic strength, salt concentration in the precipitant, temperature, gravity, and viscosity, to name but a few.

Counter-diffusion crystallization is a more recent method for obtaining protein crystals. In the counter-diffusion technique, the protein solution and the precipitant solution are juxtaposed to each other within a closed geometry, such as a capillary tube. When the two solutions diffuse against each other, a spatial-temporal gradient of supersaturation is created. As a result, the counter-diffusion technique may be used to create multiple crystallization conditions within a single capillary tube. Similar to both the batch and vapor diffusion crystallization methods, the counter-diffusion technique may require the protein solution to be screened against many different crystallization parameters.

Many protein crystallization techniques, including those discussed above, typically require that each protein solution be screened against a wide variety of different chemical and physical parameters. As a result, it should be readily apparent that obtaining protein crystals and their subsequent preparation for X-ray analysis is a very time consuming and limiting step in determining protein structure. Consequently, more efficient methods are needed for growing protein crystals suitable for analysis.

### BRIEF SUMMARY OF THE INVENTION

The present invention is directed to a device that may be useful for counter-diffusion applications and other methods for growing crystals of macromolecules, co crystallization, and/or soaking experiments. The device may be useful in the simultaneous screening of large number of compounds such as drug screening.

In one alternative embodiment, the device may comprise a removable cartridge having a plurality of capillary tubes that may be disposed between first and second members. The first member may include a reservoir tray having a plurality of reservoir wells, and the second member may include a sealant material disposed thereon. The second member may be moveable into a sealing position wherein the distal ends of the capillary tubes contact the sealant material. The first member may be moveable into first and second positions. In the first position, the proximal ends of the capillary tubes may contact a macromolecular solution, which may cause the macromolecular solution to diffuse into the interior space of the capillary tube. In the second position, the proximal ends of the capillary tubes may be inserted into a corresponding reservoir well having a precipitating solution.

The macromolecular solution and the precipitating solution may then counter diffuse against each other in each capillary tube. The removable cartridge may then be removed and replaced with a new removable cartridge. The device may be capable of automation so that the steps necessary for crystallization and drug screening may be performed with minimal handling of the samples and resulting crystals. As a result, the invention provides a means that may help improve efficiency in the screening of different crystallization conditions and drug compounds.

In another embodiment, the device is useable in batch crystallization methods. In this case, the proximal ends of the capillary tubes contact a well containing the macromolecular solution and sufficient amounts of the precipitating solution to initiate crystallization. This solution gently diffuses through the long axis of the capillary tube. The macromolecular solution may contain small molecules (i.e. substrates, co-factors, inhibitors etc.).

In some embodiments, the removable cartridge may be disposed between a sealant member that is moveable along a longitudinal pathway between a nominal position and a sealing position, and a reservoir member disposed opposite the sealant member and moveable along the longitudinal pathway between a nominal position, a first position, and a second position. In some embodiments, the reservoir member may include a reservoir tray having a plurality of reservoir wells disposed on a surface that faces the proximal ends of the capillary tubes. A precipitating solution may be disposed in the reservoir wells and a macromolecular solution, such as a protein solution, may be disposed on the surface of the reservoir tray adjacent to the reservoir wells. The sealant member may include a surface having a sealant material disposed thereon and facing distal ends of the capillary tubes.

Movement of the reservoir member into the first position may cause the proximal ends of the capillary tubes to be in fluid communication with the macromolecular solution whereby the macromolecular solution may enter the capillary tubes. Movement of the sealant member into the sealing position may cause the distal ends of the capillary tubes to contact the sealant material to seal the distal ends of the capillary tubes. Movement of the reservoir member into the second position may cause the proximal ends of the capillary tubes to be in fluid communication with the precipitating solution. The macromolecular and precipitating solution may then counter diffuse against each other to form a supersaturation wave within each capillary tube. Crystals may begin to form as the superstaturation wave moves through the capillary tube.

During the counter-diffusion process, a spatial-temporal gradient may be formed along the length of the capillary tube. Varying supersaturation conditions that lead to crystal growth may simultaneously be present in the capillary tubes. As a result, the multiple capillary tubes within each removable cartridge may improve the chances of obtaining crystals that are suitable for X-ray analysis.

In one embodiment, the removable cartridge may include one or more connectors having an engagement surface that may be capable of attaching a reservoir tray to the removable cartridge. When the reservoir member is moved into the second position, the connectors may be used to attach the reservoir tray to the removable cartridge so that the removable cartridge and the reservoir tray may be removed from the device together. As a result, once the reservoir member has been moved into the second position, the removable cartridge and reservoir tray may be removed and permitted to continue the crystallization process outside of the device.

After crystallization has been completed, the individual capillary tubes may be separated from the removable cartridge and any crystals disposed therein may be evaluated, such as with an X-ray diffractometer, in situ without having to manually handle the crystals. Alternatively, the removable cartridge may be disengaged from the initial reservoir tray and placed back in the device for another operation. For instance, a new reservoir tray may be placed in the device, e.g. with wax on the bottom of the reservoir wells and sufficient amounts of cryoprotectant in the wells. The proximal ends of the capillary tubes may then be inserted into the new reservoir tray to add additional solutions to the capillaries such as cryoprotectants, e.g. by advancing forward the capillaries to hit the wax seal in the bottom of the reservoir wells to seal the proximal ends of the capillary tubes.

In another embodiment, the device may also be useful in an application directed to the screening of drugs, heavy atom derivatives, i.e. heavy ions, or other compounds that are believed to interact with the macromolecular solution being analyzed. In this embodiment, the device may be useful to evaluate the effectiveness with which one or more compounds or heavy ions that bind to a protein or other macromolecule, such as a virus, RNA, or DNA. Further, the structures of binding complexes of the compounds or heavy atom derivatives to already crystallized macromolecules can be immediately determined in the capillaries of the removable cartridge to provide rapid visualization and knowledge of the compound binding position, or changes in the diffraction pattern as the result of the heavy atom addition, such as by X-ray crystallography.

In another embodiment, the device may also be useful in an application directed to the co-crystallization of a compound with the protein or other macromolecule of interest. In another embodiment, the device may also be useful in an application directed to soaking a protein crystal in a mother liquor that contains a ligand. Proteins can retain their crystalline state and ligands can diffuse to active and binding sites through channels of water in the crystal. Soaking performed on protein crystals with ligands is more likely to produce crystals of the same form and unit cell dimensions as those of pure proteins. In this embodiment, a liquid or solution containing a small molecular weight compound that is believed to be capable of diffusing into the macromolecular crystal, is introduced into the capillary tube and allowed to slowly diffuse into and form a complex with the macromolecular. The capillary tubes provide for relatively slow diffusion of the compound, thereby tending to reduce disruptions in lattice structure.

Thus, the invention may be a significant step forward in achieving the goal of solving the structures for thousands of macromolecules, such as proteins, and providing an efficient method of screening drugs, heavy atoms and other compounds. The device offers the advantage of screening large numbers of compounds simultaneously. Also, the concentration gradient through the length of the capillary tube provides for a continuous variation in crystallization conditions when crystallizing macromolecules and a diffusion gradient that allows for gentle infusion of a compound into a macromolecular solution during co-crystallization.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is a graphical perspective of a device that may be useful in growing macromolecular crystals and drug screening applications;

FIG. 2 is graphical perspective of the device of FIG. 1 depicting the sealant member, reservoir member, and removable cartridge separated from the device;

FIG. 3a is a graphical illustration of an embodiment of a removable cartridge;

FIG. 3b is an exploded perspective of the removable cartridge of FIG. 3a;

FIG. 4a is a graphical illustration of an alternative embodiment of a removable cartridge;

FIG. 4b is an exploded perspective of the removable cartridge of FIG. 4a;

FIG. 5a is a graphical illustration of an apparatus for correctly aligning the capillary tubes within the removable cartridge;

FIG. 5b is a graphical illustration of the apparatus of FIG. 5a in the process of aligning the capillary tubes;

FIG. 6a is a graphical illustration of a reservoir tray that may be used in the practice of the invention;

FIG. 6b is cross-sectional side view of the reservoir tray viewed along line 6b of FIG. 6a;

FIG. 6c is a graphical illustration of an alternative reservoir tray that may be used in the practice of the invention;

FIG. 7a is top view depicting the device being moved from a nominal position to a first position;

FIG. 7b is a cross-sectional side view depicting the position of the capillary tubes with respect to the reservoir wells when the reservoir member is in the first position;

FIG. 8a is a top view depicting the device being moved from a nominal position to a sealing position;

FIG. 8b is cross-sectional side view depicting the position of the capillary tubes with respect to the sealant material when the sealant member is in the sealing position;

FIG. 9a is a top view depicting the device being moved from a first position to a second position;

FIG. 9b is a cross-sectional side view depicting the position of the capillary tubes with respect to the reservoir wells when the reservoir member is in the second position;

FIG. 10 is a graphical perspective depicting the removable cartridge and an associated reservoir tray being removed from the device; and

FIG. 11 is a cross-sectional side view of the capillary tubes in fluid communication with a precipitating solution and depicting the formation of crystals in the capillary tube.

### DETAILED DESCRIPTION OF THE INVENTION

The present invention now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

Referring more specifically to the drawings, for purpose of illustration, but not of limitation, there is shown an alternative embodiment of a device for counter-diffusion applications that is designated as reference number 10. The device may include a support housing 12 having a surface 14 with a first member 22, also referred to as the “reservoir member” and a second member 24, also referred to as the “sealant member” disposed thereon. In some embodiments, the reservoir member 22 may be disposed adjacent to a proximal portion 26 of the support housing, and the sealant member 24 may be disposed adjacent to a distal portion 28 of the support housing. A longitudinal pathway 20 may extend laterally across the support housing between the reservoir member and the sealant member. A removable cartridge 30 may be disposed along the longitudinal pathway 20 between the reservoir member 22 and the sealant member 24.

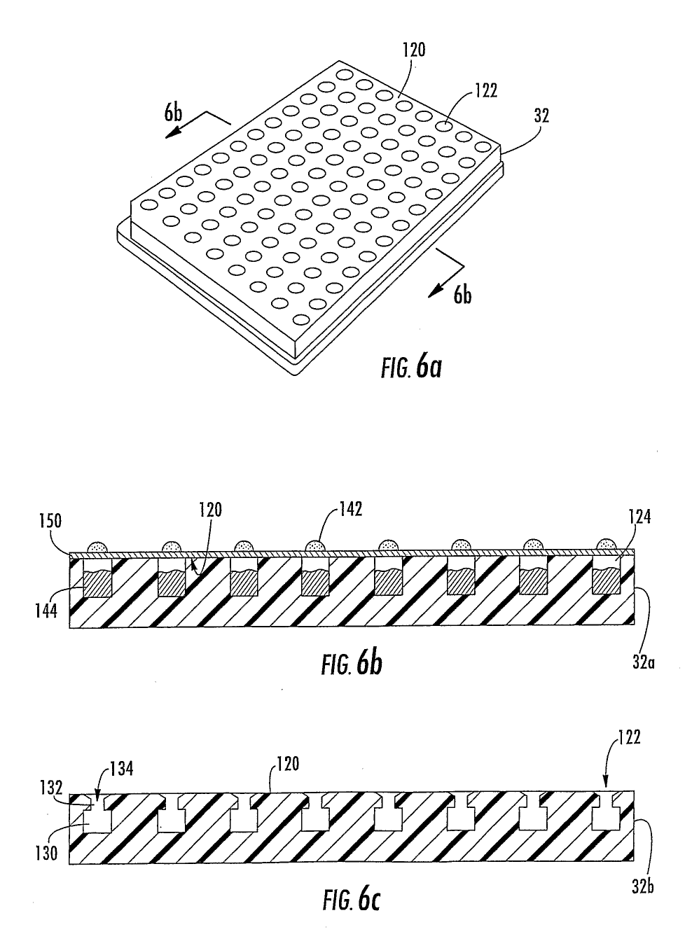

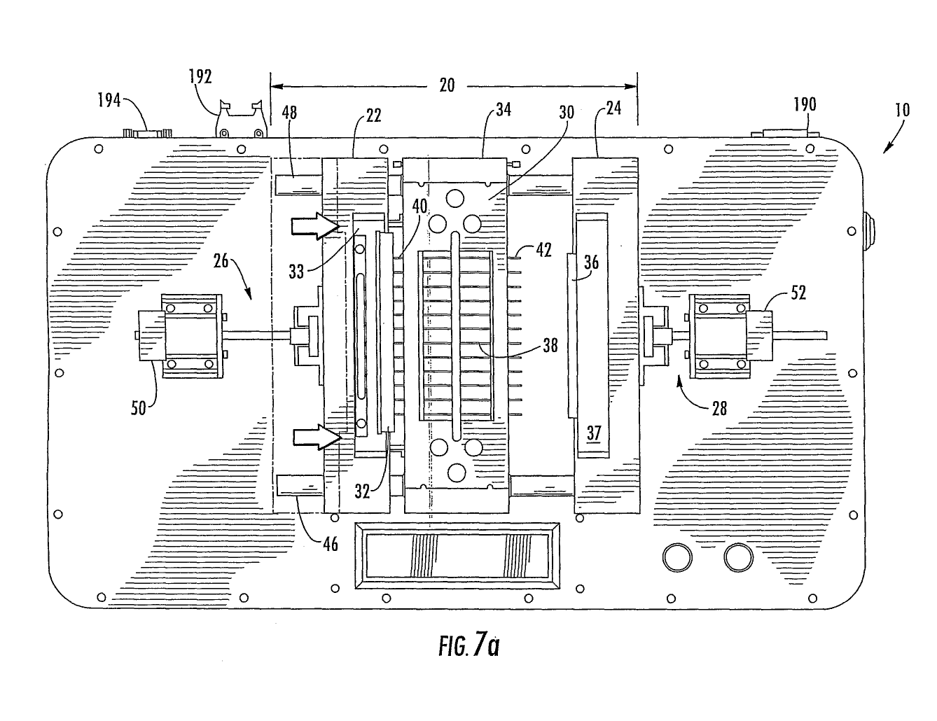

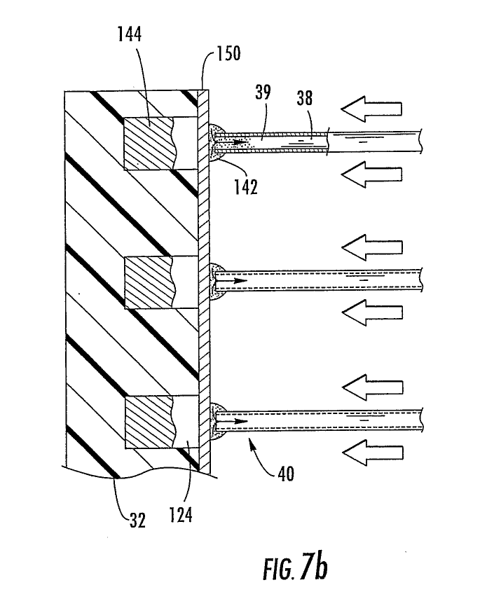

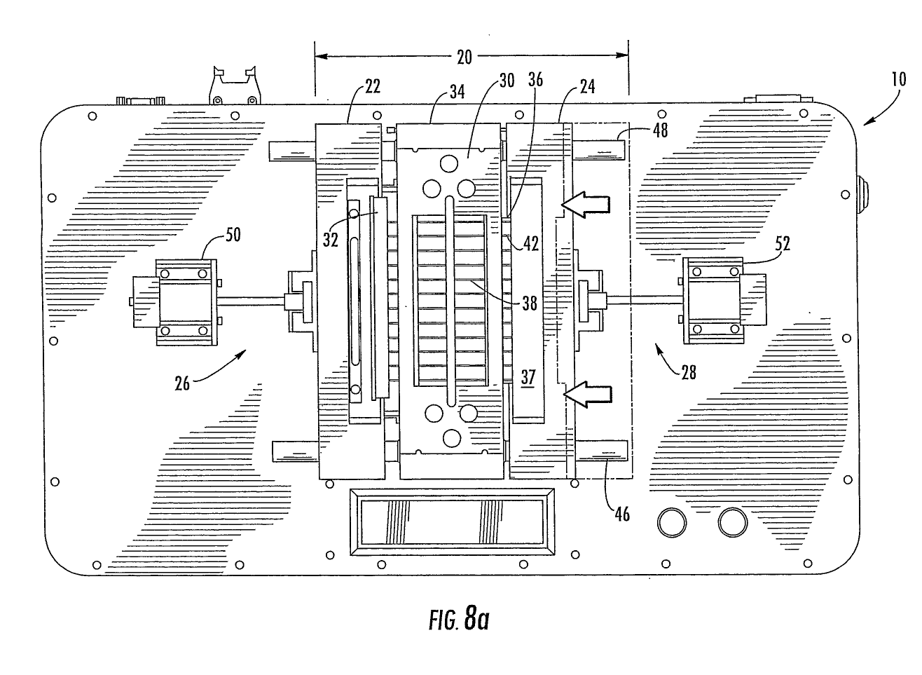

In some embodiments, a reservoir tray 32 may be disposed on the reservoir member 22. In some embodiments, the reservoir member 22 may be adapted to releasably receive the reservoir tray thereon. As discussed in greater detail below, the reservoir tray may include a surface facing in the direction of the removable cartridge and having a plurality of reservoir wells thereon (see briefly FIG. 6a, reference numbers 120, 122). Each reservoir well may have an opening aligned with the longitudinal pathway. The sealant member 24 may include a surface facing in the direction of the removable cartridge and having a sealant material 36 disposed thereon.

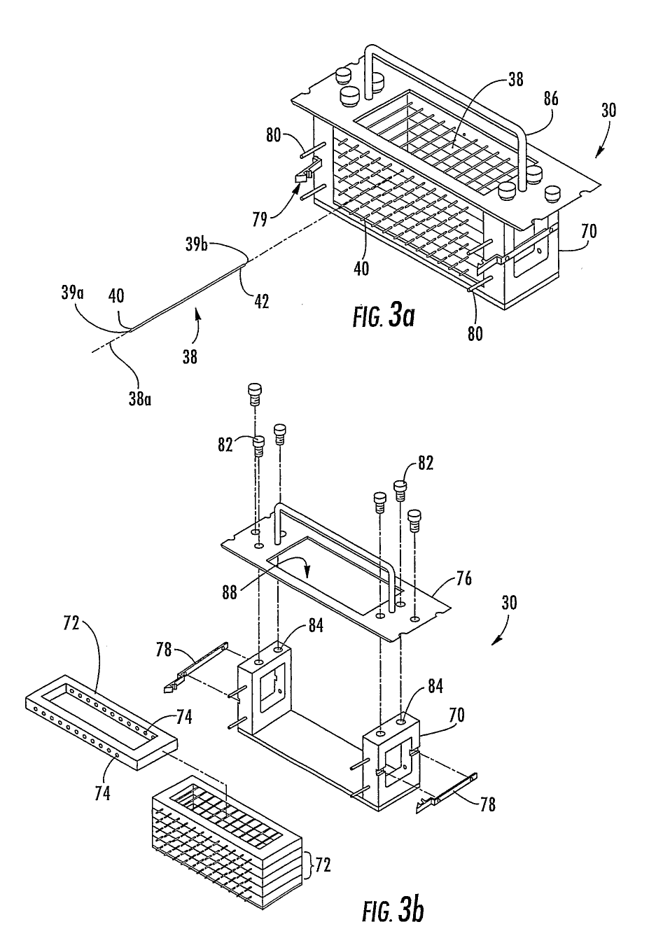

The removable cartridge 30 may include a plurality of capillary tubes 38 that each may have a proximal end 40 and a distal end 42. In some embodiments, each capillary tube may include a longitudinal axis 38a extending along the length of each capillary between proximal and distal ends 40, 42 (see briefly, FIG. 3a). The longitudinal axis may be substantially parallel to the longitudinal pathway 20. The proximal and distal ends 40, 42 of the capillary tubes may each include an opening 39a, 39b, respectively, through which a substance may pass into the interior of the capillary tubes (see briefly, FIG. 3a ). The proximal end 40 of each capillary tube may be insertably aligned with a corresponding reservoir well (not visible) disposed on a reservoir tray 32, and the distal end 42 of each capillary tube may be insertably aligned with the sealant material 36. Movement of the reservoir member in the direction of the removable cartridge, or movement of the removable cartridge in the direction of the reservoir member may cause the proximal end of each capillary tube to be removably inserted into a corresponding reservoir well. Movement of the sealant member in the direction of the removable cartridge, or movement of the removable cartridge in the direction of the sealant member may cause the distal end of each capillary tube to be removably inserted into the sealant material.

In some embodiments, the reservoir member and the sealant member may each separately be moveable along the longitudinal pathway. In other embodiments, the removable cartridge may be moveable along the longitudinal pathway.

In some embodiments, the capillary tubes may comprise an amorphous material suitable for X-ray diffraction, such as quartz or an amorphous polymer. Other materials may be used provided that they are amorphous or near amorphous and do not contribute to experimental diffraction. The size of the capillary tube may vary depending upon the intended application. The inner diameter of the capillary tube may range anywhere from about 0.01 mm to 1 mm, with capillary tubes having inner diameters less than about 0.3 mm obtaining the best results. In some embodiments, the inner diameter of the capillary tubes may range from about 0.05 to 0.4 mm. Today, due to increased intensities obtained from second and third generation synchrotron sources the majority of crystal sizes required for high-resolution structure determination fall within a range of 0.05 mm to 0.3 mm in each dimension. However, this device is broadly applicable and useful with structure determination techniques that currently exist or may be developed in the future.

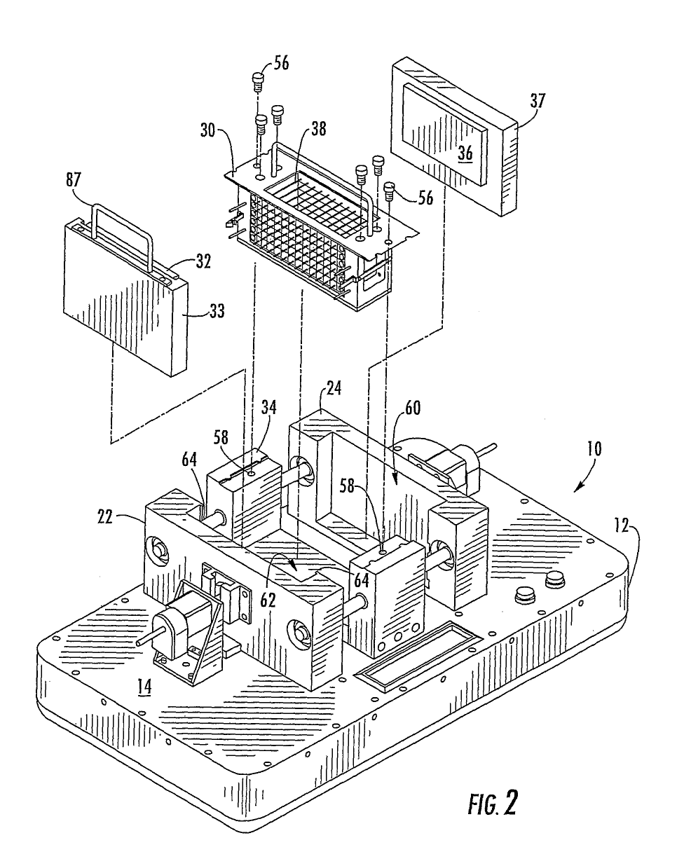

As shown in FIG. 2, the device 10 may include a cartridge housing 34 for removably receiving the removable cartridge 30 therein. The cartridge housing 34 may be disposed along the longitudinal pathway between the reservoir member and the sealant member. In some embodiments, the removable cartridge 30 may be releasably attached to the cartridge housing with one or more fasteners 56, such as a thumb screws, clips, bolts, set screws, and the like. The cartridge housing 34 may include one or more surfaces 58 that may be adapted to receive a fastener 56 therein. In one alternative embodiment, the cartridge housing may include one or more rotating clips, tabs, snaps, or the like that may be used to attach the removable cartridge to the cartridge housing, which may permit quick insertion/removal of the removable cartridge without the use of bolts, pins, or the like. In some embodiments, the one or more surfaces may comprise counter bores, counter sinks, recesses, and the like. The removable cartridge 30 may include a handle 86 to help facilitate transport and removal of the removable cartridge from the cartridge housing 34.

In one alternative embodiment, the sealant member may be moveable between a nominal position and a sealing position. In the context of the invention, the term “nominal position” refers to a position of either the reservoir member or sealant member along the longitudinal pathway wherein the reservoir member or sealant member are not in contact with the capillary tubes. In the sealing position, the distal ends of the capillary tubes may be inserted into the sealant material. In another alternative embodiment, the reservoir member may be moveable between at least a nominal position, a first position, and a second position. When the reservoir member moves into the first position, the proximal ends of the capillary tubes may contact a macromolecular solution having one or more solvated macromolecules therein. In the context of the invention “macromolecular solution” refers to a solution which may have one or more crystallizable compounds therein. Crystallizable compounds include biological or inorganic crystals, such as proteins, nucleic acids, DNA and RNA fragments, viruses, and the like; and pharmaceutical compositions, such as drug compounds, organic molecules, and the like. Contact of the proximal end of the capillary tubes with the macromolecular solution may cause the solution to diffuse into the interior space of the capillary tube. The reservoir member may be moved from the first position to the second position after a desired amount of the solution has filled the capillary tube. In some embodiments, the device may be configured to automatically move either the sealant member into the sealing position or the reservoir member at a predetermined amount of time. When the reservoir member is moved into the second position, the proximal ends of the capillary tubes may each be inserted into a corresponding reservoir well, and may be in fluid communication with a precipitating solution having one or more precipitating agents disposed therein. The macromolecular solution and the precipitating fluid may then counter diffuse against each other in the interior of each capillary tube. Typically, the sealant member is moved into the sealing position before the reservoir member is moved from the first position to the second position. In some embodiments, the reservoir member may also be moveable into a third position. In the third position, the proximal ends of the capillary tubes may contact a sealant material disposed at the base of the reservoir wells. The use of a sealant material in the reservoir wells may facilitate closure of the proximal ends of the capillary tubes so that they may be removed from the reservoir wells.

In some embodiments, the device 10 may include one or more motors 50, 52 that are operable for driving the sealant and reservoir members along the longitudinal pathway. The device may also include one or more guide rails 46, 48 that may extend laterally through one or more of the reservoir member, sealant member, and a cartridge housing. The guide rails 46, 48 may be useful for helping to facilitate alignment and travel of the reservoir member and/or sealant member along the longitudinal pathway. In some embodiments, reservoir member, sealant member, and cartridge housing may include one or more passageways that extend laterally through each member and provide a channel in which the guide rails may be disposed.

In some embodiments, the reservoir tray 32 may be attached to a tray holder 33. The tray holder may include one or more fasteners or surfaces (not visible) for attaching a reservoir tray to the tray holder. The size and shape of the tray holder may be selected to accommodate a wide variety of reservoir tray configurations. In some embodiments, the tray holder 33 may be adapted to be releasably attached to the reservoir member 22. In this regard, FIG. 2 illustrates an alternative embodiment of the invention wherein the device has a surface for releasably receiving the tray holder 33 therein. In some embodiments, the surface may comprise a slot 62 into which the tray holder 33 may be inserted. The slot 62 may include one or more outwardly extending projections 64 that may be used to releasably secure the tray holder to the reservoir member.

In some embodiments, the sealant material 36 may be disposed on a surface of the sealant member 24 that is facing in the direction of the removable cartridge 30. In some embodiments, the surface area occupied by the sealant material on the sealant member may correspond to the placement and quantity of the capillary tubes within the removable cartridge so that the distal ends of the capillary tubes may be removably inserted into the sealant material. The sealant material 36 may comprise a material that is suitable for sealing closed an opening of the capillary tube and does not adversely interfere with the growth of macromolecules therein. Suitable sealant materials include various waxes, synthetic or natural, clays, and combinations thereof.

In one alternative embodiment, the sealant member 24 may include a removable sealant receptacle 37 having a surface upon which the sealant material 36 may be disposed. In some embodiments, the sealant member may include a surface 60, such as a slot for receiving the sealant receptacle therein. The sealant receptacle may be attached to the sealant member with one or more fasteners, clips, frictional fittings, slots/grooves, and the like, and combinations thereof. In one embodiment, the surface 60 may include one or more outwardly extending projections (not shown) that may be adapted to engage and secure the sealant receptacle to the sealant member.

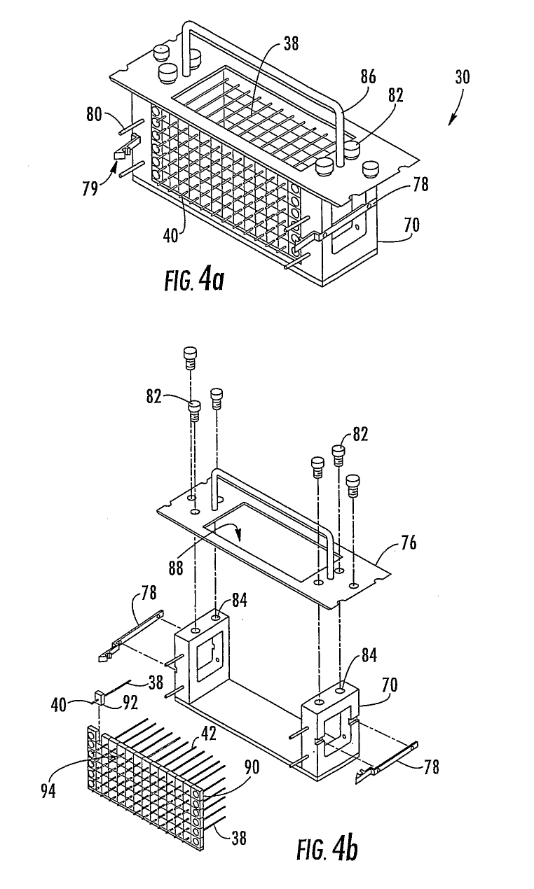

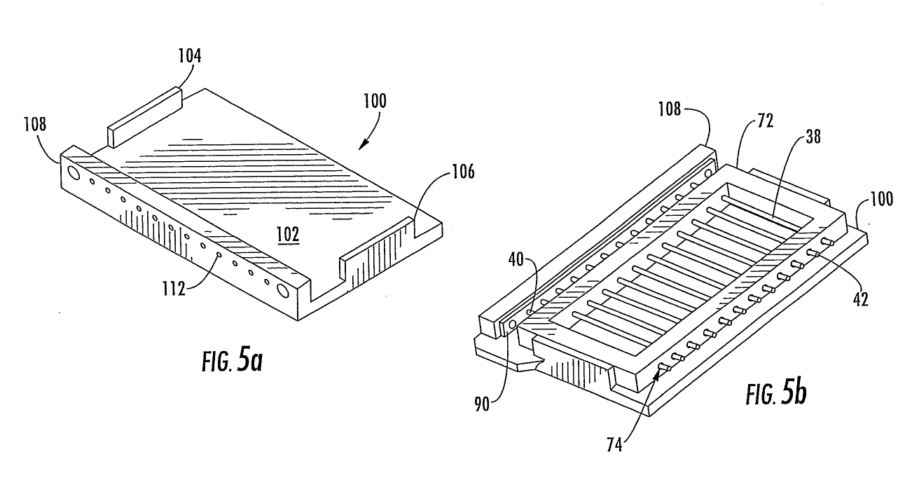

With reference to FIGS. 3a and 3b, an alternative embodiment of the removable cartridge 30 is illustrated. In FIG. 3b an exploded perspective of the removable cartridge of FIG. 3a is illustrated. In some embodiments, the removable cartridge 30 may include a carriage assembly 70 having a plurality of carriage frames 72 vertically stacked one on top of the other. The carriage frames may include one or more capillary tube passageways 74 that extend laterally through the carriage frame 72. Each capillary tube passageway may be capable of receiving and securing a capillary tube therein. The proximal and distal ends of each capillary tube may extend outwardly from the capillary tube passageways 74. In some embodiments, the number and placement of each capillary tube passageway may be configured to correspond to the placement and number of reservoir wells on the reservoir tray. In some embodiments, one or more capillary tube retainers (see briefly FIG. 4a, reference number 90) may be used in conjunction with a carriage frame. In this regard, FIG. 3a illustrates an embodiment wherein a capillary tube retainer may be attached to the carriage frame 72 with one or more screws. The carriage assembly may also include one or more inwardly extending lips (not visible) that hold the carriage frames within the carriage assembly.

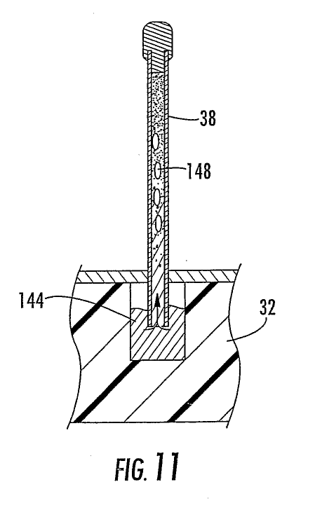

In some embodiments, the removable carriage may also include an upper plate 76 that may be attached to the carriage assembly. The upper plate 76 may include a handle 86 to help facilitate removal and transport of the removable cartridge. The upper plate may be attached with screws 82 that are insertable into a corresponding bore 84 on the carriage assembly. The upper plate may also include an opening 88 through which the capillary tubes may be visualized. In some embodiments, the removable cartridge 30 may include one or more connectors 78 that may be adapted to releasably engage a reservoir tray disposed on the reservoir member. The carriage assembly 70 may also include one or more projections 80 that extend outwardly in the direction of the reservoir member. Projections 80 may be insertable into a corresponding recess disposed on the reservoir member and/or tray holder. For example, movement of the reservoir member in the direction of the removable cartridge may cause each projection 80 to engage and be inserted into the corresponding recess disposed on the reservoir member and/or tray holder. Continued movement in the direction of the removable cartridge may cause connectors 78 to engage the reservoir tray or the tray holder (see briefly FIG. 2, reference number 33). In some embodiments, the connectors 78 may include a snap-like structure 79 that engages and grips the backside of the tray holder. As a result, the tray holder and the removable cartridge may be joined together and can be removed from the device as a single unit (see briefly FIG. 10).

With reference to FIGS. 4a and 4b, an alternative embodiment of the removable cartridge is illustrated. FIG, 4b is an exploded perspective of the removable cartridge of FIG. 4a. In this embodiment, in place of a carriage frame, the removable cartridge includes a plurality of capillary tube retainers 90 vertically stacked one on top of the other within the carriage assembly. In some embodiments, the capillary tube retainer 90 may comprise a flexible strip having one or more passageways through which one or more capillary tubes may be inserted. The capillary tube retainer may include a score line 94 between each passageway that permits a section 92 and an associated capillary tube to be separated from the capillary tube retainer 90. The score line typically comprises a line of weakening that is formed in the capillary tube retainer and extends laterally across the width of the capillary tube retainer. In some embodiments, the score line can be created by cutting a recess into the capillary tube retainer that extends partially through the width of the capillary tube retainer. In other embodiments, the score line may comprise an intermittent line of weakening having a plurality of spaced recesses or slits that may extend through the full width of the capillary tube retainer. In some embodiments, the separated section and capillary tube may be directly mountable in an instrument, such as an X-ray diffractometer, for in situ analysis of capillary contents. X-ray data obtained from this configuration can be used for ab initio structure determination where diffraction data may be implemented interactively for electron density calculations.

In some embodiments, an individual row of capillary tubes and an associated row of reservoir wells may be removable from the remaining rows of capillary tubes and reservoir wells. For example, FIGS. 3a and 3b illustrate removable cartridge that may include a carriage assembly 70 having a plurality of carriage frames 72. In one embodiment, an individual carriage frame and an associated row of reservoir wells may be removable from the remaining carriage frames and reservoir wells. Removing a carriage frame and the associated reservoir wells may help facilitate visualization of any crystals that may be growing in the capillary tubes. In some embodiments, an individual capillary tube and its associated reservoir well may be removable. Similarly, an individual capillary tube retainer 90 depicted in FIGS. 4a and 4b and its associated reservoir wells may also be removable from the plurality of capillary retainers.

In another embodiment, unique codes or symbology may be used to uniquely identify the individual capillary tubes. In this embodiment, unique codes or symbology may be assigned and secured to the tube retainers or the capillary tubes. These codes or symbols may be alpha/numeric, graphical, bar or the like and may be used for sample, solution or experiment tracking. These codes or symbols may be inscribed into or onto the capillary tube retainer or capillary tube by the use of a laser etching device or another device or may be attached to the retainer or capillary tube by the use of adhesive backed labels or the like.

With reference to FIGS. 5a and 5b an apparatus 100 that may be used to align the capillary tubes within carriage frame and/or the capillary tube retainer is illustrated. The apparatus 100 may be used to help ensure that a desired portion of the proximal ends of the capillary tubes may extend outwardly from the capillary tube retainer. As a result, when the sealant member is moved into the first position, the proximal portion of each capillary tube may be in fluid communication with a macromolecular solution disposed adjacent to the opening of a reservoir well. Apparatus 100 may include a surface for receiving an assembly frame 72 or capillary tube retainer 90 thereon. In some embodiments, the apparatus 100 may include side members 104, 106 for positioning the carriage frame on surface 102. The apparatus 100 may include a surface 108 having a plurality of passageways 112 that may be alignable with the capillary tube passageways on the carriage frame or the capillary tube retainer. In some embodiments, the capillary tubes may be aligned by inserting them through the capillary tube passageways and the passageways 112 on surface 108 until the proximal ends of the capillary tubes are correctly aligned with the surface 108. In some embodiments, the capillary tubes may be securely attached to the capillary tube passageways with a suitable glue or epoxy. In other embodiments, the capillary tubes maybe securely attached to the capillary tube passageways via a frictional fitting.

With reference to FIG. 6A, an exemplary reservoir tray 32 that may be used in the practice of the invention is illustrated. In one alternative embodiment, the reservoir tray 32 may include a surface 120 having a plurality of reservoir wells 124 disposed thereon. In some embodiments, the reservoir wells 124 define a recess in the surface of the reservoir tray which may be adapted to have one or more precipitating agents disposed therein. The surface of the reservoir tray may also include a plurality of openings 122 through which a capillary tube may be inserted into the reservoir well. The size and structure of the reservoir tray may be varied depending upon a preference of a user. In some embodiments, the sealant member may be adapted to be used with a wide variety of both commercially available and custom-made reservoir trays. For example, suitable reservoir trays may have from as little as one reservoir well to in excess of 500 reservoir wells. In one alternative embodiment, the reservoir tray may have for example, 84, 96, 240, 384 reservoir wells or greater.

As discussed above, the reservoir tray 32 may be disposed on the reservoir member so that surface 120 faces in the direction of the removable cartridge. In some embodiments, the openings 122 may be insertably aligned with a corresponding capillary tube disposed within the removable cartridge. As shown in FIG. 6b, the reservoir tray may include a sealing layer 150 for sealably containing one or more precipitating agents within the reservoir wells. The sealing layer may function as a membrane to seal the precipitating solutions in the reservoir wells while at the same time allowing the capillaries' proximal ends to penetrate and enter the reservoir wells. In some embodiments, a macromolecular solution 142 may be disposed on the surface of the sealing layer 150 adjacent to the openings of the reservoir wells. As discussed in greater detail below, when the reservoir member is moved into a first position, the proximal ends of the capillary tubes may contact the macromolecular solution causing at least a portion of the macromolecular solution to enter and fill the interior space of the capillary tubes. In a subsequent step, the reservoir member may then be moved into a second position. Movement into the second position may cause the proximal ends of the capillary tubes to pierce the sealant layer and pass into the interior space of the reservoir well. The proximal ends of the capillary tubes may then contact one or more precipitating agents disposed within the reservoir well. The one or more precipitating agents may then counter diffuse against the macromolecular solution disposed in each capillary tube.

The sealant layer may include any material that does not adversely affect the one or more precipitating agents that may be disposed in the reservoir wells and that may be piercable by the proximal ends of the capillary tubes. In some embodiments, the sealing layer may comprise a material that may be able to create a sealing relationship between the capillary tube and sealing layer after the capillary tubes have pierced the sealant layer. Having a tight seal between the sealant layer and the capillary tube may be desirable to prevent evaporation of the fluid in which the one or more precipitating agents may be disposed. Suitable materials for the sealing layer include, but are not limited to, polyethylene films, latex, plastic plugs, waxes, agarose, fracture ease, and combinations thereof. In one alternative embodiment, the sealing layer may comprise a wax or other material that may be disposed at the opening of each reservoir well. In one embodiment, a piercable sealing tape, such as Advanced Piercable Polyethylene Tape, available from NUNC may be used.

FIG. 6b is a cross-sectional side view of the reservoir tray 32a viewed along line 6b of FIG. 6a. FIG. 6b depicts a reservoir tray 32b that has been prepped and is ready for immediate use in the device. As seen in FIG. 6b, a user has deposited a droplet 142 of macromolecular solution on the sealant layer 150 above the opening of each reservoir well. The reservoir wells include a fluid 144 having one or more precipitating agents disposed therein. In some embodiments, the fluid 144 may be in the form of a gel-like fluid. In some embodiments, the fluid may also include a scattering atom component, cryoprotectant, ligands, drug compounds and or additional components that may help facilitate the growth and analysis of macromolecular crystals within the capillary tubes. In one alternative embodiment, the reservoir wells may include a sealant material (not shown) that may be deposited in the base of the well before any fluid or precipitating agent has been deposited in the well.

The precipitating agent may contain one or more salts (e.g. ammonium sulfate, sodium chloride or sodium citrate at concentrations of about 2-3M), alcohols (e.g. ethanol, proponal, methylpentanediol at concentrations of about 35-75%), or different forms of polyethylene glycol (PEG) (e.g. PEG 4000,6000,8000 concentrations between 15-50%) in a buffered media, or combinations thereof. In some embodiments, the volume of the precipitating solution (including any additional additives) placed into the cavities can be as little as the equivalent volume of the protein solution contained within the capillary.

Some protein crystals may be sensitive to X-rays and in some cases may not survive the X-ray exposure that is necessary for data collection. As a result, in some cases it may be desirable to super-cool the crystals in preparation for X-ray analysis. Preferably, the crystals may be super-cooled without allowing the solvent content within the capillary tube to go through an ice transition before X-ray analysis. In some embodiments, cooling of the crystals may be accomplished by subjecting the crystal to a stream of cryogenic vapor (with temperatures around −150° to −170° C.), such as that from liquid nitrogen. For the sake of simplicity, the supercooled crystals will be also referred to as frozen crystals. In order for the crystal to endure the cooling process it may be treated with a cryoprotectant prior to freezing. This could be accomplished two ways, by initially adding the cryoprotectant to the precipitating solution deposited into the reservoir wells or by adding the cryoprotectant after crystallization has occurred by reinserting the capillary tubes into a separate reservoir tray that contains any of a variety of cryoprotectants. Capillaries can then be sealed for analysis. The cryoprotectant solution may protect the crystal while still sustaining its ability to diffract X-rays. Examples of cryoprotectants may include glycerol, multiple alcohols, polyethylene glycols, oils, and even Indian cooking butter. Useful oils may include glycerides of the fatty acids, such as oleic, palmitic, stearic, and linolenic. Other chemical substances can be used provided that they sufficiently protect the crystal's structure during freezing, there is no resulting interference with crystal nucleation, and the crystal's ability to diffract X-rays is not adversely affected.

In some embodiments, to grow macromolecular crystals adequate for ab initio phase determination, including isomorphous replacement and anomalous scattering types of phasing, it may be desirable to find a strong scattering atom that is intrinsic to the macromolecules or to incorporate a derivative scattering atom, such as a heavy metal or halide into the crystal. Bromide and iodide are halides that have been shown to be useful for diffusing into protein crystals and have been successfully used in crystallographic phasing. The anomalous X-ray scattering signals of halides may be strong enough to provide phase information for X-ray crystallography, and as such, may be useful for incorporation into the macromolecular solution.

In some embodiments, the reservoir trays may be available preloaded and sealed with a desired precipitating agent disposed in the reservoir wells. The preloaded reservoir trays may each include a variety of different precipitating agents to help screen which precipitating agents facilitate crystal growth. In some embodiments, reservoir trays may be manufactured and pre-loaded with varying precipitation solutions. This may permit a researcher to select and purchase reservoir trays that are ready for installation into the device without requiring the need to transfer solutions into the individual reservoir wells.

In FIG. 6c an alternate embodiment of the reservoir tray is illustrated. In this embodiment, the reservoir tray 32b may include an opening 122 that may have a conical shaped recess 134 that may be in fluid communication with a reservoir well 130 via a narrow channel 132. In some embodiments, the narrow channel 132 may be slightly larger than the diameter of a corresponding capillary tube. This may be useful in applications where it may be desirable to use capillary tubes having diameters on the order of 0.01 mm or less. The narrow channel 132 may help secure and direct the capillary tube into the interior space of the reservoir well. In some embodiments, the conical-shaped opening 134 provides a surface into which a macromolecular solution may be deposited. In this embodiment, the reservoir tray optionally does not include a sealing layer as the narrow channel 132 may provide the desired separation between the macromolecular solution and the precipitating solution.

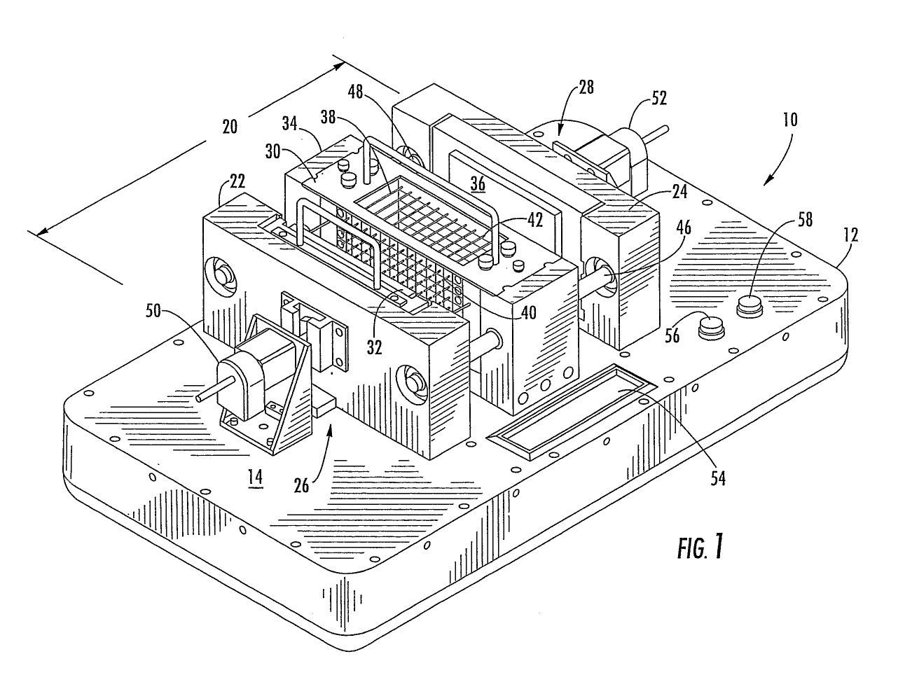

In some embodiments, the device may include a control unit (not shown), such as a microprocessor, that may be used to control the various operational states, such as positioning the members of the device. In one embodiment, the control unit may include a programmable logic controller (PLC) that may be operable to control the timing and sequence of each step in the operation. Referring back to FIG. 1, the device 10 may also include one or more buttons 56, 58 that may be capable of instructing the device to perform one or more operations, such as positioning the members. In some embodiments, the device may include a user interface in the form of a touch pad (not shown) that may be used to select among various menu options and to input operational commands into the device. In other embodiments, the device may also include a display 54, such as an LCD display, that may be capable of communicating information to a user, such as the current operational state of the device, remaining time for any given operation, future operational states and the like. In some embodiments, the device may be pre-programmed to include one or more crystallization methods, such as vapor diffusion or batch, that may be selected by a user. In some embodiments, the device may also include one or more limit switches (not shown).

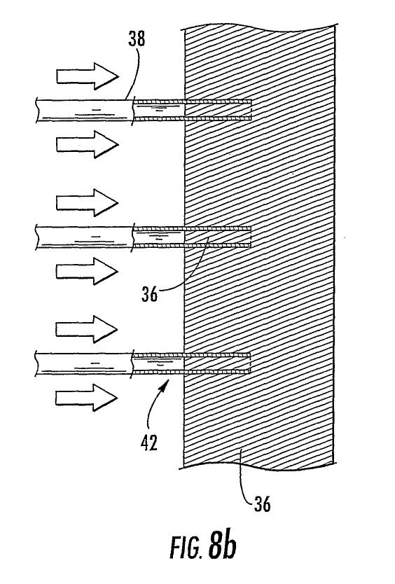

The device may include one or more input/output interfaces that may facilitate communication between the device and an external computer. In this regard, FIG. 7a depicts a device 10 that may have one or more I/O interfaces 192, 194. The I/O interfaces may comprise wired or wireless connectivity means such as I2C, ACCESS.bus, RS-232, universal serial bus (USB), IEE-488(GPIB), LAN/Internet protocols such as TCP/IP, wireless means such as infrared (IR) communication, 802.11x, and Bluetooth, etc. In some embodiments, the I/O interface may comprise a combination of wired and wireless connectivity means. The I/O interfaces may facilitate communications with an external computer or with one or more devices. In some embodiments, the I/O interface may be used to remotely control the device from an external computer. The device may also include a power adapter 190 for connection to an external power source.

With reference to FIGS. 7a through 9b, a process of growing macromolecules using the device of the invention is illustrated. In FIG. 7a the reservoir member 22 is depicted in the process of being moved between a nominal position and a first position. In the context of the invention the term “first position” refers to the position of the reservoir member with respect to the proximal ends of the capillary tubes wherein the capillary tubes are in fluid communication with a macromolecular solution whereby the macromolecular solution may transfer into the capillary tubes. In some embodiments, when the reservoir member is moved into the first position, the proximal ends 40 of the capillary tubes approach the surface of the reservoir tray 32 and may contact a macromolecular solution disposed adjacent to an opening of a reservoir well. In this regard, FIG. 7b illustrates the proximal ends 40 of the capillary tubes 38 contacting a macromolecular solution 142. In some embodiments, the macromolecular solution 142 may be disposed on a sealing layer 150 that may cover a surface of the reservoir tray 32. One or more precipitating agents 144 may be disposed in the interior space of a reservoir well 124. Contact of the capillary tube and the macromolecular solution 142 may cause the macromolecular solution 142 to enter and at least partially fill the interior 39 of the capillary tube. Optionally, the device may have an automated delay sequence to allow for macromolecular solution 142 to be wicked.

The sealant member may be moved into a sealing position after a sufficient amount of macromolecular solution has filled the capillary tubes. FIG. 8a illustrates the sealant member 24 being moved from a nominal position to a sealing position. In the context of the invention the term “sealing position” refers to the position of the sealant member with respect to the distal ends of the capillary tubes wherein the distal ends are in contact with the sealant material so that the sealant material may sealably close the distal ends of the capillary tubes. In this regard, FIG. 8b illustrates the distal ends 42 of the capillary tubes 38 contacting a sealant material disposed on the sealant member. Movement into the sealing position causes the distal ends 42 to enter into the sealant material. As a result, a portion of the sealant material is forced into and enters the distal ends of the capillary tubes resulting in the sealing of the tubes. Closure of the distal ends of the capillary tubes helps to create a closed environment.

In one embodiment, sealant material may be disposed on the bottom of the reservoir walls and the proximal end of the capillaries may be sealed by contacting the proximal end 40 of the capillaries with the sealing material after the macromolecular solution 142, precipitating solution, or other solution has been allowed to transfer into the capillary. Any time delay before contacting the proximal end 40 of the capillary with the sealant may be manual or automated.

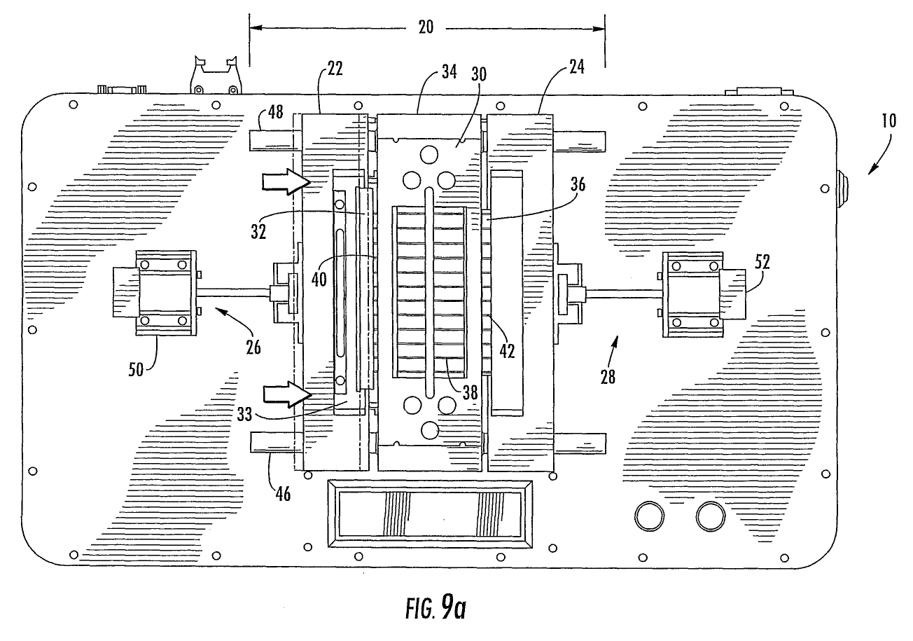

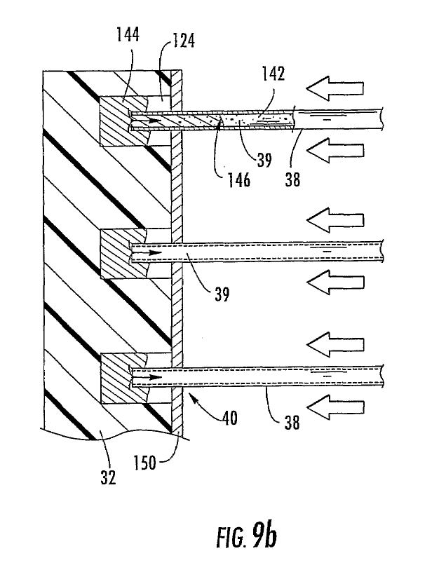

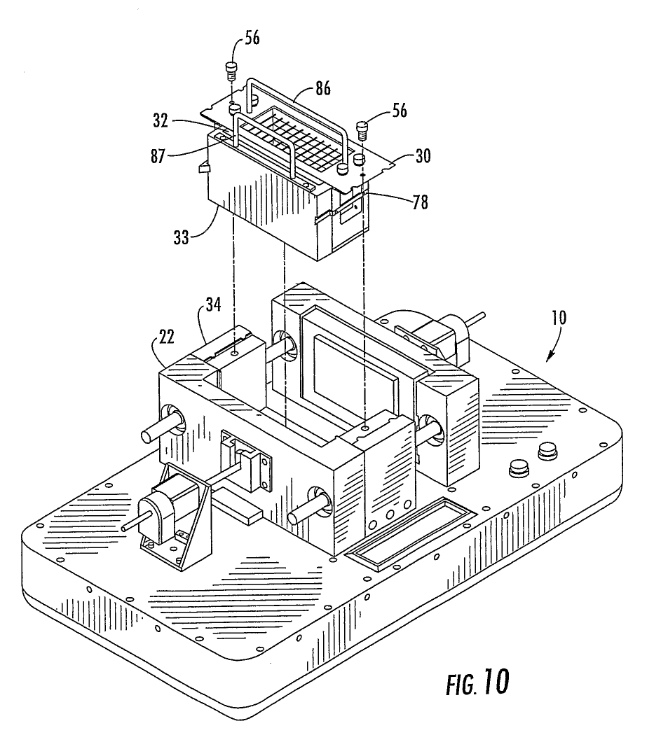

In a subsequent step, the proximal ends of the capillary tubes may be moved into a second position. FIG. 9a illustrates the reservoir member 22 moving into the second position. In the context of the invention the term “second position” refers to the position of the reservoir member with respect to the proximal ends of the capillary tubes wherein the capillary tubes are disposed in the interior space of the reservoir well and are in fluid communication with a precipitating solution disposed therein. In this regard, FIG. 9b illustrates the proximal ends of the capillary tubes in fluid communication with a precipitating solution 144. When the precipitating solution 144 (salt solution) contacts the macromolecular solution 142, a liquid-liquid counter-diffusion system is formed activating a super saturation wave along the capillary. This gradient is a result of the precipitating solution initially diffusing into the protein solution, forming a gradient of high concentration near the protein-precipitating interface 146 and falling to a lower concentration as it moves across the capillary. As a result of the gradient, crystallization conditions may not be uniform throughout the capillary and crystals of varying quality and size may be produced. Thus, one advantage of the device is that multiple crystallization conditions may be present in a single capillary tube. The amount of time for equilibration of the two solutions and for crystal growth may be varied depending on the preferences of a user and/or the particular crystallization screens being conducted. In some cases, it may be desired to allow crystallization to proceed for at least two weeks. After a desired amount of time has passed, the capillary tubes may be screened for possible crystal growth and may be subject to additional analysis.

In some embodiments, when the reservoir member is moved into the second position, the one or more connectors (see briefly FIG. 3b, reference number 78) may engage and secure the reservoir tray to the removable cartridge. Thereafter the removable cartridge and the reservoir tray may be removed from the device to allow further equilibration between the macromolecular solution and the precipitating solution. In this regard, FIG. 10 illustrates an embodiment wherein the reservoir tray 32 and/or the tray holder 33 and the removable cartridge 34 are secured together and may be removed from the device to permit continued crystallization of the macromolecules outside of the device. The device may now be ready to repeat the process and a new reservoir tray, tray holder, and removable cartridge may be inserted into the device. As a result, the device may be used to conduct multiple crystallization screenings without having to wait for the completion of crystal growth within the capillary tubes secured in an individual cartridge. In embodiments where the reservoir member is movable into a third position, the proximal ends of the capillary tubes may be moved into the third position when it is desirable to seal the proximal ends of the capillary tubes. In some embodiments, this may occur at a predetermined time or after crystals are observable in the capillary tubes.

FIG. 11 depicts one or more crystals 148 in the process of growing in the interior of the capillary tube 38. In some embodiments, single crystals may be observable within 3 to 7 days of equilibration. In embodiments, the crystals may be sufficiently cryoprotected within 1 to 4 weeks of equilibration depending on the cryoprotectant and macromolecular solutions. As discussed above, the invention provides a means wherein equilibration and/or crystallization may be completed outside of the device. As a result, the use of a removable cartridge and reservoir tray may help facilitate the screening of thousands of crystallization conditions in a cost-efficient and productive manner.

The device may be particularly useful in processes involving high throughput crystallization techniques. Automated systems may be used to take the device through one or more operations making it possible to perform tests on thousands of samples simultaneously. For instance, numerous reservoir trays may be manufactured and pre-loaded with varying precipitation solutions. This may permit a researcher to select and purchase reservoir trays that are ready for installation into the device without requiring the need to transfer precipitating solutions into the reservoir wells. The device may be used to screen a given macromolecule against multiple crystallization conditions simultaneously. As a result, the device may be used to efficiently determine the optimal conditions for crystallization.

Another application of the device involves high throughput screening for protein function. The advent of genomics and high throughput proteomics has resulted in the discovery and production of thousands of new proteins. Unfortunately, very little is known about the function of many of these proteins that are being produced from eukaryotic and prokaryotic organisms as well as viruses. The device can be used to rapidly screen a protein against a variety of other proteins, substrates or cofactors, whereby binding of any of these molecules to the protein could be optically monitored through the capillaries via colorimetric/spectroscopic techniques.

In some embodiments, the device may be adaptable to a robotic system that could deposit a predetermined amount of macromolecular solutions onto the surface of a preloaded reservoir tray, load the reservoir tray into the reservoir member, and take the device through the steps necessary for macromolecular crystallization by repositioning the reservoir and sealant members at predetermined time intervals. After completing the crystallization steps, the automated system may be adapted to remove the removable cartridge from the device to permit further equilibration and crystal growth. A new removable cartridge and reservoir tray may then be placed into the device and the process repeated.

A further advantage of the device is that it may be used to simultaneously combine the processes of crystallization, high X-ray scattering atom incorporation, and cryoprotection. This may permit the crystals to remain in a stable environment at all times and eliminates the need for physical manipulation or exposing the crystals to drastic chemical changes. After crystal growth is completed the crystals may be evaluated in situ without ever having to remove them from their original growth environment.

In another alternative embodiment, the device may also be useful for the screening of drugs, heavy atom derivatives, or other compounds. In this embodiment, the device may be useful to evaluate the effectiveness with which one or more compounds bind to a protein or other macromolecules. The reservoir wells may contain one or more compounds that may counter-diffuse against a protein solution in the capillary tubes, during or after crystallization. As a result, the use of a removable cartridge and reservoir may permit the determination of the binding ability of thousands of drug candidates or other compounds that can be revealed by in situ techniques, such as X-ray diffraction. Further, the structures of binding complexes of the compounds to already crystallized proteins can be immediately determined in the capillaries of the removable cartridge to provide rapid visualization and knowledge of the binding position in the protein or other macromolecules by techniques of structure determination, such as X-ray crystallography. The device may also be useful to evaluate the binding of pharmaceutical compounds and other compounds with non-protein pathogenic molecules, such as viruses, RNAs, and DNAs.

In another embodiment, the device may also be useful in an application directed to the co-crystallization of a compound with a protein or other macromolecule of interest. In this embodiment, a liquid or solution containing a small molecular weight compound is prepared with the macromolecular solution introduced into the capillary tube and allowed to slowly form a complex. Crystallographers have traditionally been required to co-crystallize proteins with many (50 or more) different small molecules (compounds) or one or more other proteins.

In another embodiment the method for soaking crystals of the original protein in a stable solution that includes the compound(s). The hope is that the compound will slowly diffuse into the crystal and form a complex with the protein. It is important that the compound diffuse slowly into the crystal since rapid diffusion can often disrupt the lattice structure of the crystal leading to poorer quality complex crystals. In some cases rapid diffusion can even cause cracks within the crystals. The device offers the advantage of naturally slowing the compound diffusion rate due to the fact that the compound has to diffuse from the reservoir through a portion of the capillary before reaching the crystal. A natural gradient of the compound concentration would occur down the long axis of the capillary thereby providing a gentle infusion of the compound into the crystal.

In another embodiment, for vapor diffusion, one could arrange the device such that the capillaries, after penetrating the seal of their respective reservoir wells do not come in contact with the reservoir or precipitating solution, thus creating an air gap between the proximal end of the capillary and the precipitating solution, allowing diffusion of the precipitating solution to occur.

In another embodiment, for batch methods the initial protein droplets would be deposited into the reservoir wells that may contain a sealant material in the bottom and contain sufficient amounts of precipitating agents that may promote crystallization. The proximal ends of the capillaries would be disposed into the second position to penetrate the sealing layer and enter into the solution and fill the capillaries. The proximal ends of the capillary may be sealed with the sealant material.

It is evident from the foregoing discussion, that the device may be a beneficial tool to a crystallographer. The design of the device may facilitate testing multiple precipitating solutions and crystallization conditions simultaneously. The removable cartridge and reservoir tray may help facilitate high-throughput crystallization processes. The removable cartridge may also permit the device to be used continuously for crystallization screening processes without having to wait until crystal growth is completed before beginning a new crystallization screen. In some embodiments, the device may be adaptable to automated processes from the initial crystallization steps to the analysis procedures performed on an X-ray diffractometer. As such, the device may be a valuable tool that may aid crystallographers in deciphering and solving the structures for thousands of macromolecules.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A device for counter-diffusion applications, said device comprising:
a first member moveable along a longitudinal pathway between at least a nominal position, a first position, and a second position, said first member having a first surface for receiving a reservoir tray thereon;
a second member disposed opposite said first member and moveable along said longitudinal pathway between a nominal position and a sealing position, said second member having a second surface for receiving a sealant material thereon; and
a removable cartridge disposed on said longitudinal pathway between said first and second members, said removable cartridge having a plurality of capillary tubes disposed therein, each of said capillary tubes having a proximal end aligned with said first surface and a distal end aligned with said second surface.

2. The device according to claim 1, further comprising a reservoir tray disposed on said first member, said reservoir tray having a plurality of reservoir wells disposed on a surface thereof, said reservoir wells each having an opening that is substantially aligned with said longitudinal pathway.

3. The device according to claim 2, wherein said reservoir tray includes a precipitating solution disposed in at least one of said reservoir wells and a sealing layer covering the openings of said reservoir wells to thereby enclose said precipitating solution within said reservoir wells.

4. The device according to claim 2, wherein said reservoir tray includes a macromolecular solution disposed in at least one of said reservoir wells and a sealing layer covering the openings of said reservoir wells to thereby enclose said precipitating fluid within said reservoir wells.

5. The device according to claim 3, wherein a macromolecular solution is disposed on an outer surface of said sealing layer opposite said openings.

6. The device according to claim 5, wherein the proximal ends of said plurality of capillary tubes are in fluid communication with said macromolecular solution when the first member is in the first position.

7. The device according to claim 5, wherein the proximal ends of said plurality of capillary tubes are in fluid communication with said precipitating solution when the first member is in the second position.

8. The device according to claim 2, wherein the first member is moveable into a third position, and wherein the proximal ends of the capillary tubes are in contact with a sealant material disposed in the reservoir wells when the first member is in the third position.

9. The device according to claim 2, wherein the precipitating solution comprises one or more precipitating agents, cryoprotectant, atom scattering component, or combinations thereof.

10. The device according to claim 5, wherein the macromolecular solution comprises biological or inorganic crystals, proteins, nucleic acids, DNA, and RNA fragments, viruses, pharmaceutical compositions or combinations thereof.

11. The device according to claim 2, wherein said reservoir tray includes from about 24 to 384 reservoir wells.

12. The device according to claim 2, wherein said reservoir tray includes a corresponding reservoir well for each of said plurality of capillary tubes.

13. The device according to claim 1, further comprising a sealant material disposed on said second surface, and wherein the distal ends of said plurality of capillary tubes are in contact with said sealant material when the second member is in the sealing position.

14. The device according to claim 1, wherein the removable cartridge further comprises one or more connectors having at least one engagement surface for securing a reservoir tray to said removable cartridge when the first member is in the second position, whereby the removable cartridge and a secured reservoir tray are removable from said device.

15. The device according to claim 1, wherein the capillary tubes are separable from or adjustable with respect to the removable cartridge, thereby allowing x-ray or other analysis of the capillary contents.

16. A device for counter-diffusion applications, said device comprising:
a reservoir member disposed at a proximal portion of a longitudinal pathway;
a reservoir tray disposed on said reservoir member and having a plurality of reservoir wells disposed on a surface thereof, said reservoir wells each having an opening that is substantially aligned with said longitudinal pathway;
a sealant member disposed at a distal portion of said longitudinal pathway and having a sealant material disposed thereon; and
a removable cartridge disposed between said reservoir member and said sealant member within said longitudinal pathway, said removable cartridge having a plurality of capillary tubes disposed therein, each of said capillary tubes having a proximal end insertably aligned with a corresponding reservoir well on said reservoir tray and a distal end insertably aligned with said sealant material, and
wherein movement of either said reservoir member or said removable cartridge towards one another along said longitudinal pathway causes the proximal ends of the capillary tubes to be removably inserted into a corresponding reservoir well, and movement of either said sealant member or said removable cartridge towards one another along said longitudinal pathway causes the distal ends of the capillary tubes to be removably inserted into said sealant material.

17. The device according to claim 16, wherein said removable cartridge is non-moveable along said longitudinal pathway.

18. The device according to claim 16, wherein said reservoir tray includes a sealant layer covering the openings of said reservoir wells.

19. The device according to claim 16, wherein the device includes one or more motors for moving the reservoir member and the sealant member along the longitudinal pathway.

20. The device according to claim 16, further comprising a controller that is operable for controlling the functions of said device.

21. The device according to claim 16, wherein said sealant material comprises a wax, clay, or a combination thereof.

22. The device according to claim 16, wherein the removable cartridge includes at least 96 capillary tubes and the reservoir tray includes at least 96 reservoir wells.

23. The device according to claim 16, wherein one or more pharmaceutical compositions are disposed in an interior of said reservoir wells.
